(19) United States
(12) Patent Application Publication
(10) Pub. No.: US 2024/0055087 A1
(43) Pub. Date: Feb. 15, 2024

(54) SYSTEM AND METHOD OF INTEGRATING TEXT MESSAGING NOTIFICATION FEATURES WITH AN EMR SYSTEM

(71) Applicant: Teledact Inc., Thornhill (CA)

(72) Inventor: Tommy Tsz Him CHEUNG, Thornhill (CA)

(21) Appl. No.: 17/886,508

(22) Filed: Aug. 12, 2022

(51) Int. Cl. G16H 10/65; H04W 4/12; G06F 40/58

(52) U.S. Cl. CPC G16H 10/65; H04W 4/12; G06F 40/58

(57) ABSTRACT

A system and method for integrating a text messaging notification system with an electronic medical record (EMR) system. A messaging notification system that provides for secure text messaging to a mobile phone is integrated with an EMR system wherein the text messages are stored as medical records in the EMR system. No data is saved on the message notification system but stored directly in the EMR system. Two-factor authentication and security features are also implemented to ensure secure authentication of the patient. The EMR system can be configured to provide multi-lingual and localization message support, and integration with chatbot, calendar and scheduling features.

### CROSS REFERENCE TO RELATED APPLICATIONS

The application claims priority to and the benefit of US Utility Provisional Application Ser. No. 63/232,427, entitled “SYSTEM AND METHOD OF INTEGRATING TEXT MESSAGING NOTIFICATION FEATURES WITH AN EMR SYSTEM”, filed on Aug. 12, 2021, US Utility Provisional Application Ser. No. 63/232,424, entitled “SYSTEM AND METHOD OF INTEGRATING TEXT MESSAGING READ NOTIFICATION WITH AN EMR SYSTEM”, filed on Aug. 12, 2021, and U.S. application Ser. No. 17/662,486, entitled “SYSTEM AND METHOD OF INTEGRATING TEXT MESSAGING READ NOTIFICATION WITH AN EMR SYSTEM”, filed on May 9, 2022, both of which are incorporated herein by reference in their entirety.

### BACKGROUND

The embodiments described herein relate to telemedicine, in particular technologies related to integrations with EMR systems.

Electronic medical records (EMR) are used by doctors and clinician offices to store patient medical information including individual contact information, and patient medical treatment and history. Currently, some EMR systems, such as Oscar Pro EMR do not have a built-in communication system, such as an instant messaging or text messaging system to communicate with patients. Further these systems also lack the ability to securely store messages in each patient's medical chart.

It is time-consuming for staff at a clinic or doctor's office to call or email and subsequently manage the calls or emails for updates, missed appointments, rescheduling, and other administrative tasks. Furthermore, manually updating these interactions in the patient's medical record is labor-intensive. This typically requires logging into the software, searching for the patient chart, entering the update, saving, and logging out.

With the Greater Toronto area being a multicultural city, there are large numbers of patients from different backgrounds and thus many are not fluent in English. A multilingual messaging tool that provides automatic translation would be highly useful. Some patients only have access to a land-line telephone, and this makes it difficult to constantly manage phone calls to them.

There is a desire to integrate a secure text notification system with an EMR system. There is also a desire to ensure a secure channel of communication and desire for multilingual support.

### SUMMARY

A system and method for integrating a text messaging notification system with an electronic medical record (EMR) system. A messaging notification system that provides for secure text messaging to a mobile phone is integrated with an EMR system wherein the text messages are stored as medical records in the EMR system. No data is saved on the message notification system but stored directly in the EMR system. Two-factor authentication and security features are also implemented to ensure secure authentication of the patient. The EMR system can be configured to provide multi-lingual and localization message support, and integration with chatbot, calendar and scheduling features.

### BRIEF DESCRIPTION OF THE DRAWINGS

FIG. 1 is a block diagram illustrating an exemplary EMR notification system.

FIG. 2 is a diagram illustrating the functionality of a messaging system with an EMR system.

FIGS. 3A and 3B are diagrams illustrating sending a messaging via a portal.

FIGS. 4A and 4B are diagrams illustrating receiving a message on a phone.

FIGS. 5A to 5D are diagrams illustrating review of a patient chart on an EMR system.

FIG. 6 is a diagram illustrating a further exemplary EMR notification system.

FIGS. 7A to 7J are diagrams illustrating an exemplary mobile device translation workflow.

### DETAILED DESCRIPTION

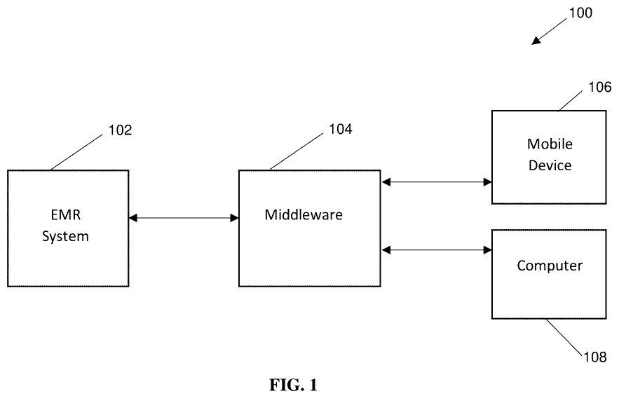

FIG. 1 is a block diagram illustrating an exemplary EMR notification system. According to FIG. 1, EMR notification system 100 consists of an EMR system 102 such as Oscar Pro used to store patient electronic medical records (EMR). A software middleware layer 104 connects to the EMR system 102 and connects to one or more mobile device 106 or computer 108 to provide EMR notification messages. The notification messages can be email, SMS text messages or voice calls.

According to FIG. 1, the software middleware layer 104 includes application programming interface (API) connections to different messaging protocols, that enable the EMR system 102 to interface to applications on the mobile device 106 and computer 108. According to further aspects of FIG. 1, the software middleware layer 104 will be a tool that enables different stakeholders to communicate. For example, the middleware layer 104 will enable patient to communicate with a coordinator, a coordinator to communicate with a pharmacy, and a coordinator to communicate with a physician.

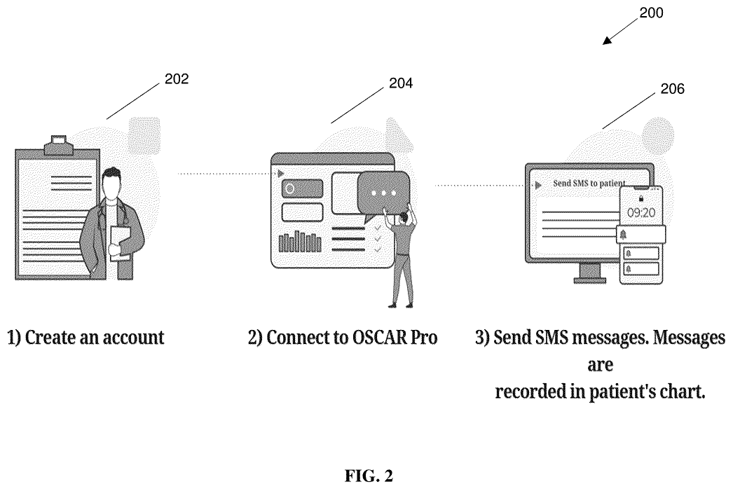

FIG. 2 is a diagram illustrating the functionality of a messaging system with an EMR system. According to FIG. 2, users of the clinic or doctor's office would create a patient account at step 202 in the EMR system 200. The user would then connect to the EMR system such as Oscar Pro at step 204. Thereafter, the user would send a short message service (SMS) text message to the patient's phone at step 206. The SMS message will be recorded in the patient's chart or record at step 206 within the EMR system 206.

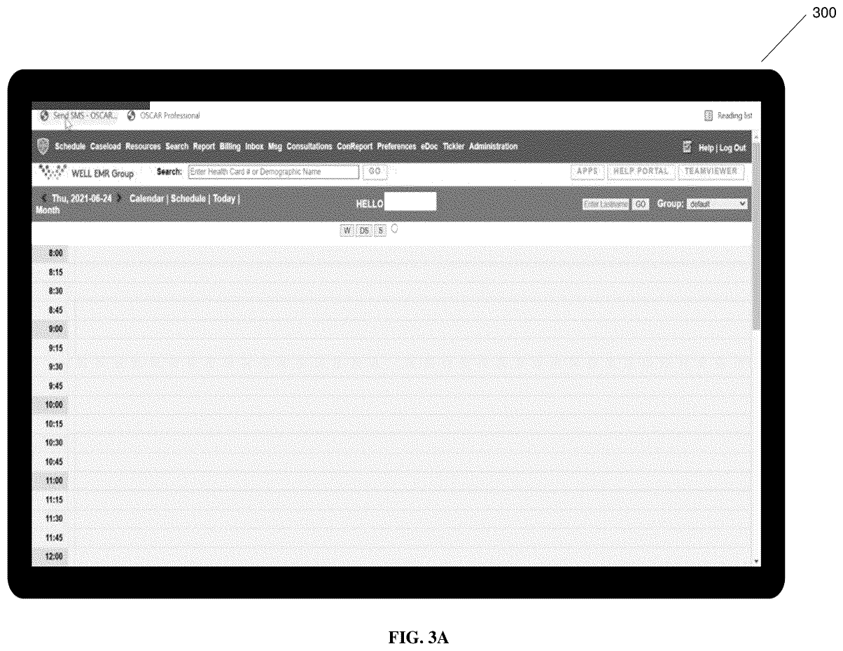

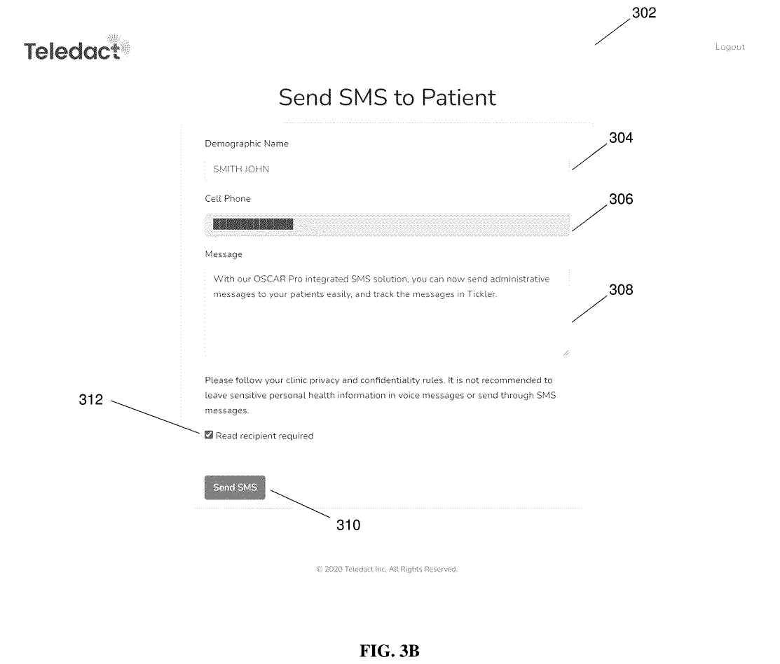

FIGS. 3A and 3B are diagrams illustrating sending a messaging via a portal. According to FIG. 3A, a messaging portal 300 is shown. A user or staff would log onto the EMR system and select the selection “Send SMS”. According to FIG. 3B, a Send SMS to Patient user interface 302 is shown. A message is sent from user or staff to patient from a portal page 302 where the staff enters patient name 304, mobile phone number 306 (e.g., cell phone field) and then type out a message 308. Thereafter, the message is sent to the patient (recipient) once the Send SMS button is selected. A further option box 312 to request read receipt is also provided which will allow the system to provide notification whether the message is read if this feature is supported (i.e., for mobile devices). In further embodiments, the mobile phone number field 306 may be replaced with a landline telephone number, email, voicemail, instant message, or translation service.

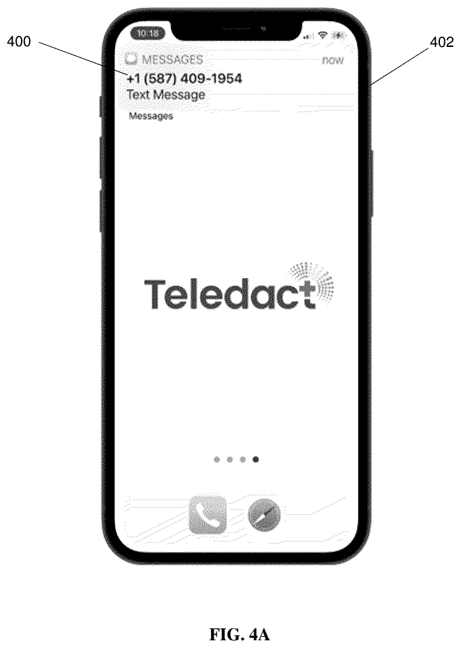

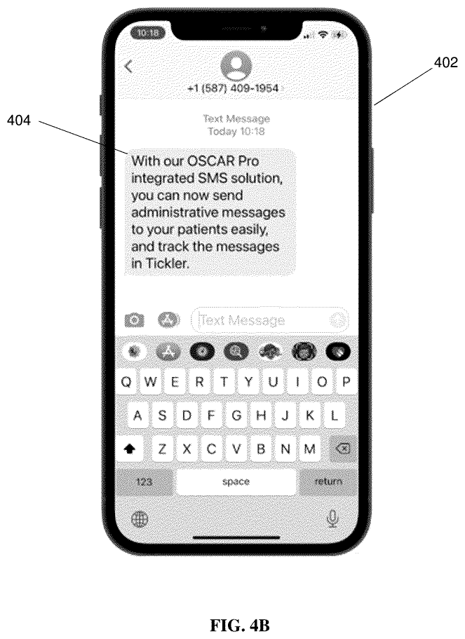

FIGS. 4A and 4B are diagrams illustrating receiving a message on a mobile phone. FIG. 4A, shows a text message (i.e., SMS message) notification 400 on a mobile phone 402. FIG. 4B illustrates the text message 404 received on the mobile device. In addition to received SMS message on a mobile phone, the message can also alternatively be sent to an email, an instant message service, a landline phone or to voice mailbox.

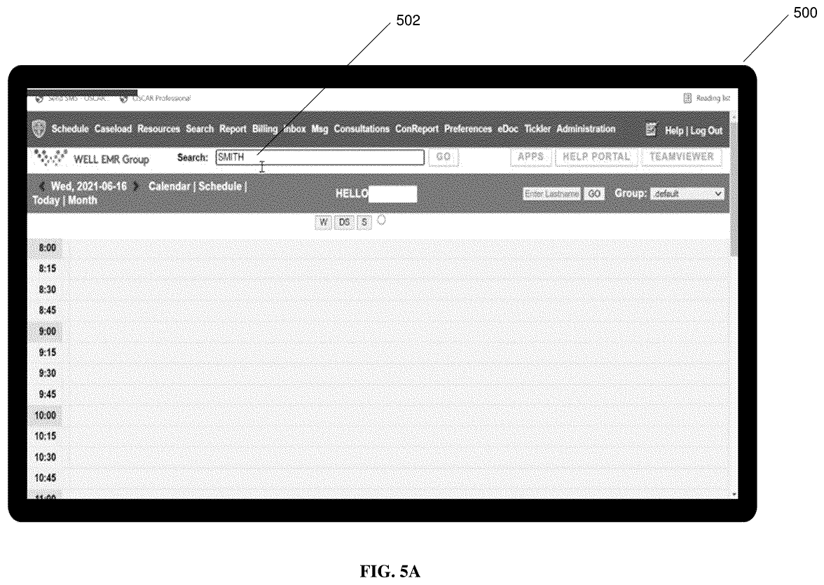

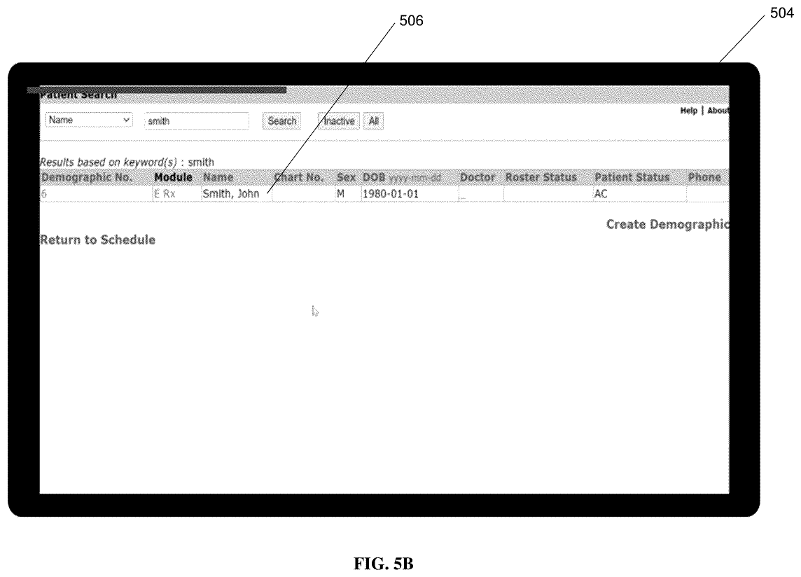

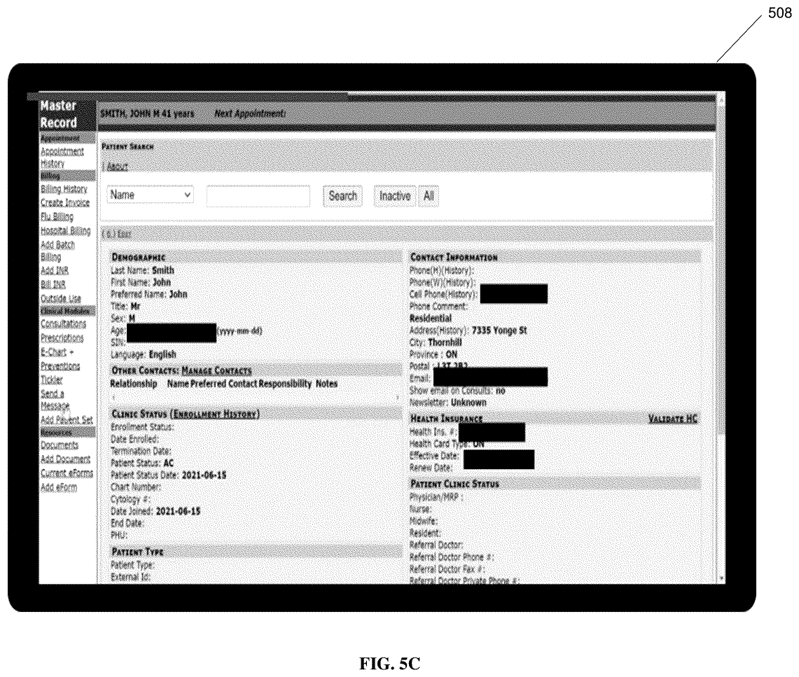

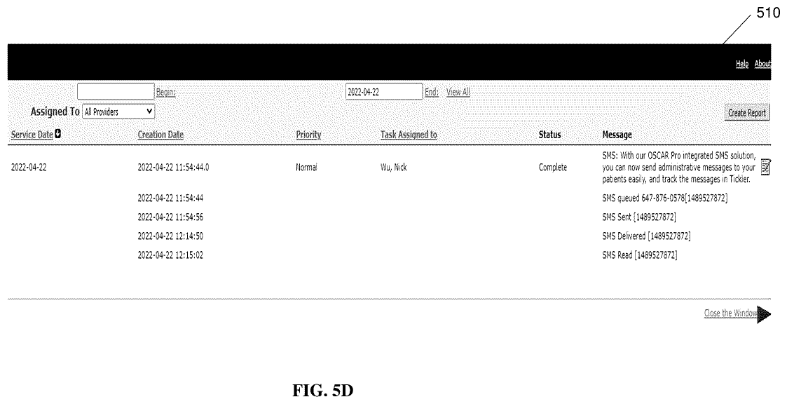

FIGS. 5A to 5D are diagrams illustrating review of a patient chart on an EMR system. FIG. 5A illustrates exemplary dashboard 500 of an EMR system. The user types in a patient name, for example “Smith” in the Search bar 502. FIG. 5B is a diagram illustrating the search results for “Smith” 504. As shown in FIG. 5B, one record for “John Smith” 506 is retrieved. FIG. 5C illustrates the master record for the patient “John Smith” 508. FIG. 5D is a further screenshot of the master record illustrating recorded SMS messages 510. As seen in FIG. 5D, the SMS message sent to the mobile phone (FIG. 4A and FIG. 4B) are stored within the EMR system and can be retrieved and displayed to the user. Text and email messages are automatically (and permanently) recorded in the patient's medical chart.

In a further embodiment, SMS communication will reduce missed appointments and return the lost hours (from calling/email patients) back to clinic staff with the ability to quickly communicate with patients via SMS messages. The messages sent to patients are automatically recorded in an EMR system (i.e., Oscar Pro) medical chart without the need for staff to manually updating them.

According to further embodiments, messages created from the portal page can be sent as a text message. Furthermore, message can alternatively be sent as an email or can be texted to the landline to users without a mobile phone and only access to a landline. There messages are also automatically updated in the patient's chart in an EMR system.

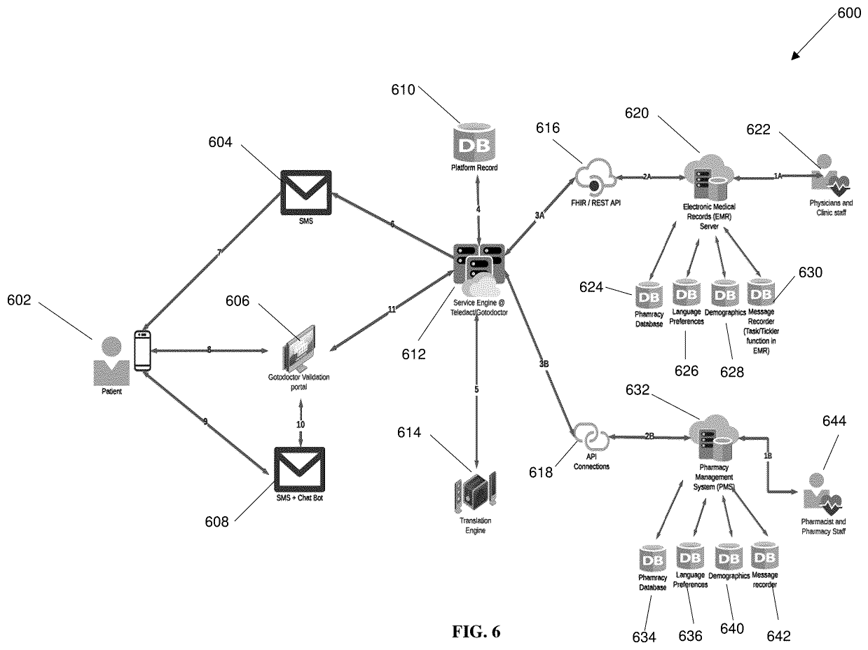

FIG. 6 is a diagram illustrating a further exemplary EMR notification system. According to FIG. 6, EMR notification system 600 consists of a service engine 612 on the internet cloud. Domains for service engine may include “Teledact”, “Gotodoctor” and other external facing internet domains. Service engine 612 is connected to translation engine 614, platform database 610 and validation portal 606. Validation portal 606 may include a “Gotodoctor” domain portal. Service engine 612 utilizes short messaging system (SMS) 604 and SMS and Chat box 608 to communicate with the mobile device of the patient 602.

According to FIG. 6, service engine 612 utilizes the Fast Healthcare Interoperability Resources (FIHR) application program interface (API) and/or Representation State Transfer (REST) application program interface (API) 616 to communicate with one or more Electronic Medical Records (EMR) server 620. FIHR is a standard describing data formats and elements and an application programming interface (API) for exchanging electronic health records. The Representational State Transfer (REST) is a software architectural style that describes a uniform interface between decoupled components on the Internet in a Client-Server architecture.

According to FIG. 6, EMR server 620 enables connection and communication with physicians and clinic staff 622. EMR Server also enables connective to other databases including pharmacy database 624, language preferences database 624, demographics database 628 and message recorder database 630. Message recorder database 630 also functions as a task or ticker function in the EMR system.

According to FIG. 6, service engine 612 also utilized other application programming interface (API) connections to interface with a pharmacy management system (PMS) 632. Pharmacy management system (PMS) 632 enables connection and communication with physicians and clinic staff 644, as well as connectivity to other relevant databases. These relevant databases may include a pharmacy database 634, language preferences database 636, demographics database 640 and message recorder database 642

### Translation Engine and Multi-Lingual Localization

In further embodiments of this disclosure, the EMR system and SMS notification system may be combined for translation with the option of multi-lingual localization capabilities. Messages created from the EMR portal, in English, can be received by patients as a bilingual text or voice message (English and 1 other language). There is also an option for patients to choose one language of their choice (i.e., French, German, Dutch, Spanish, Mandarin Chinese, Cantonese Chinese, Punjabi, Japanese, Vietnamese, etc.). Once the message is entered and stored in English text, it can be sent to a translation engine whereupon it can be machine translated into another text message and/or verbally recorded into a voice message in a second language, whereupon it can be delivered in the second language via SMS text message, email message or “.wav” file as a voice note.

### Chat Bot

In further embodiments, the EMR system may integrate with an artificial intelligence (AI) chat bot. For example, a user clicks selects a SMS message on a mobile device. The user clicks on a “book appointment” link. An AI bot will reply automatically and help the patient to book an appointment via SMS (e.g., send # to book).

### System Workflow

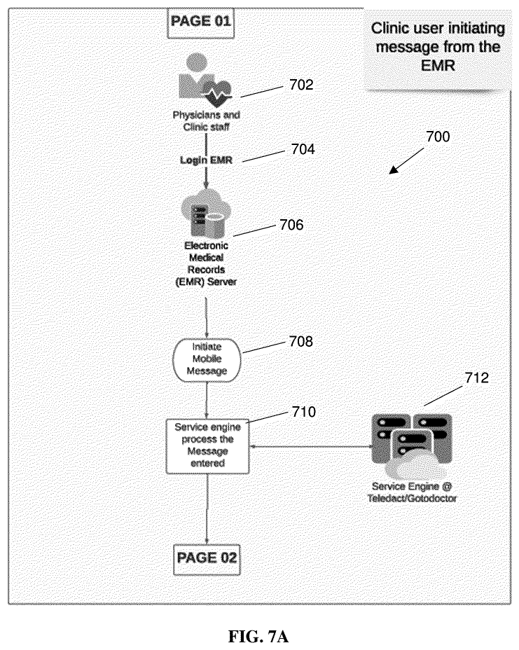

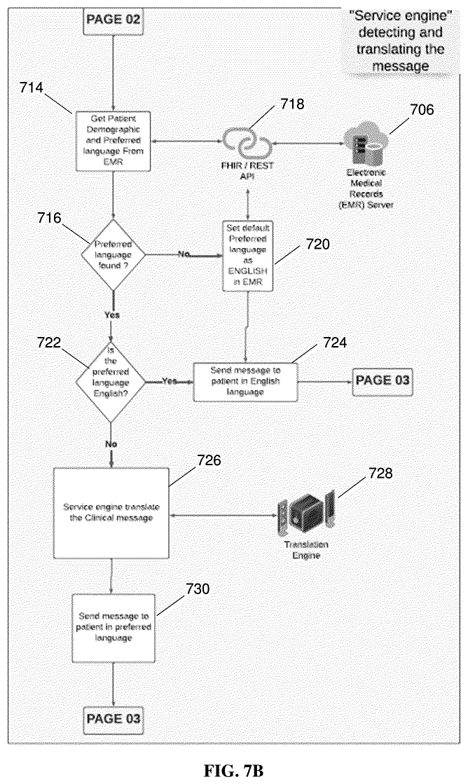

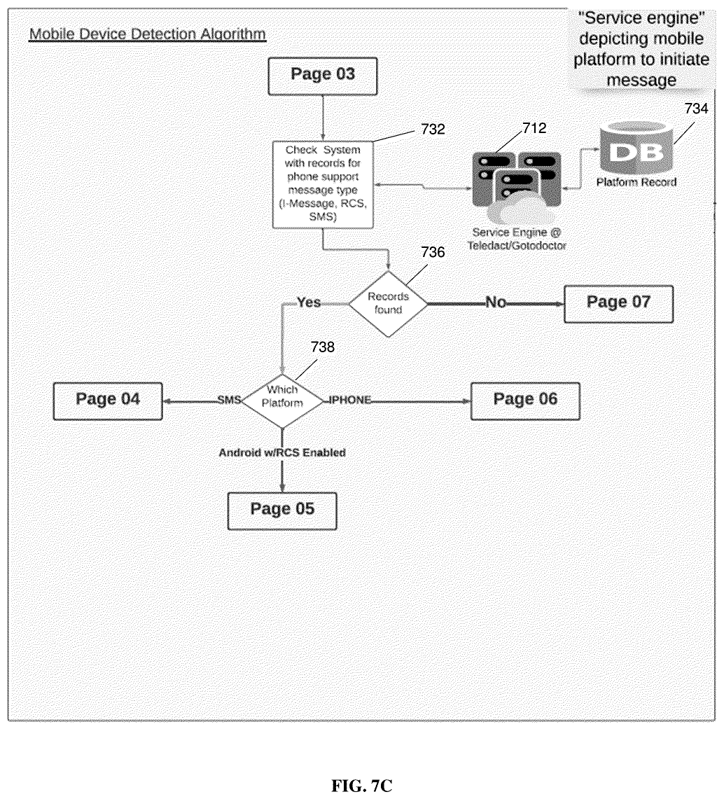

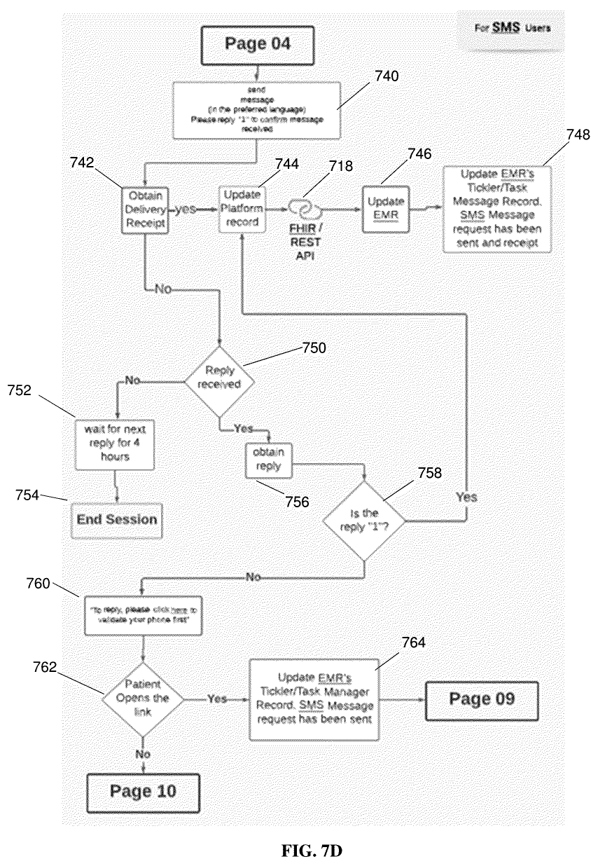

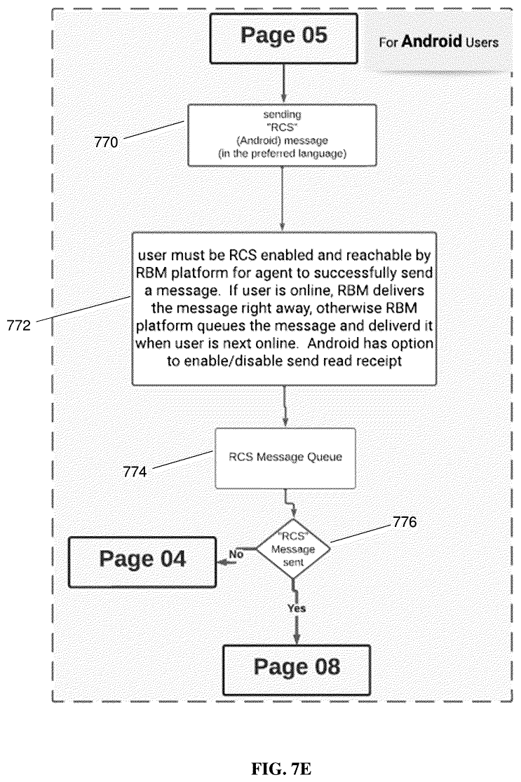

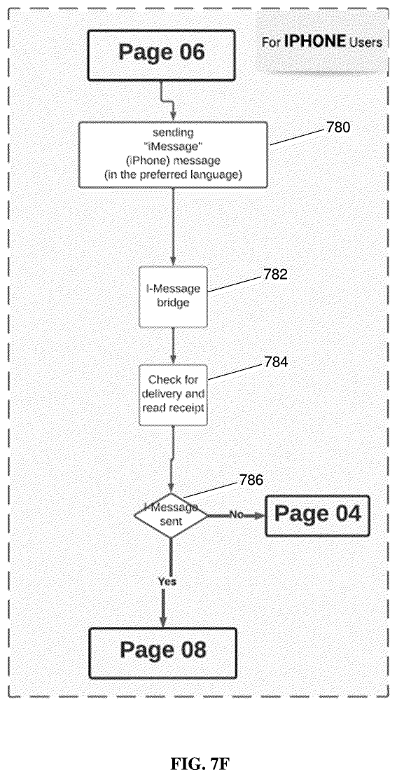

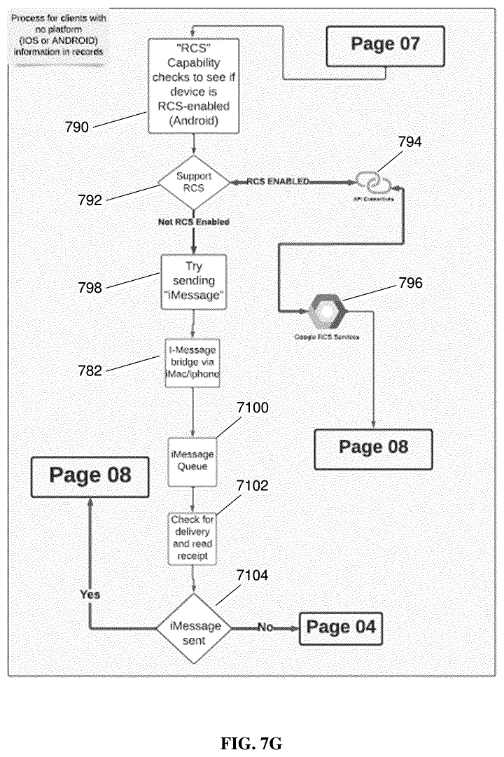

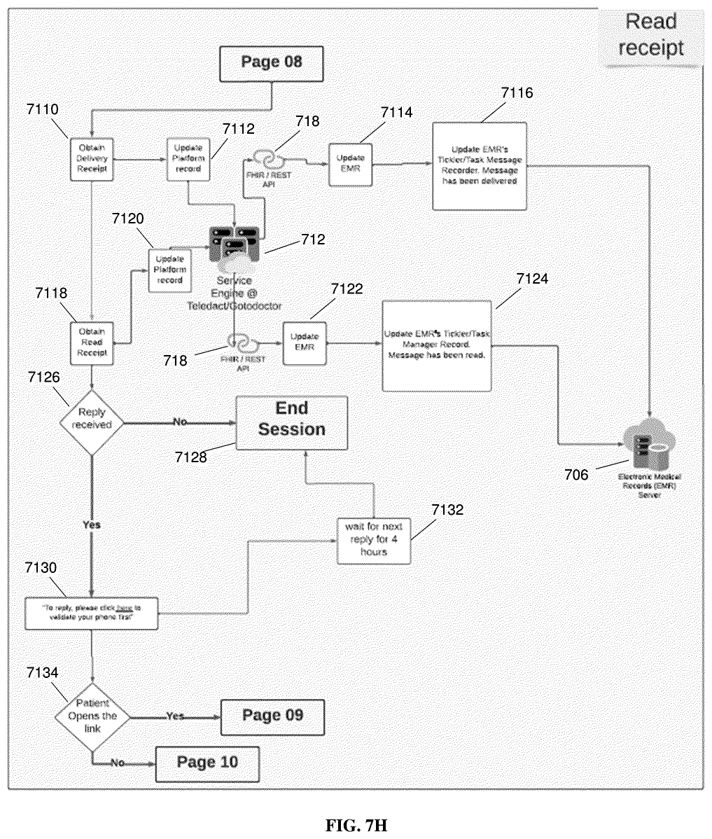

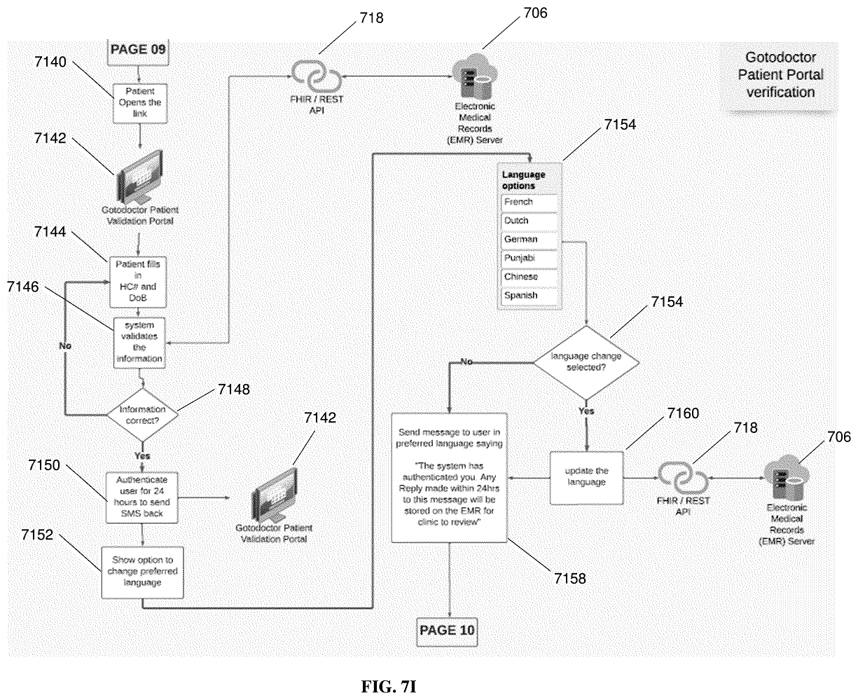

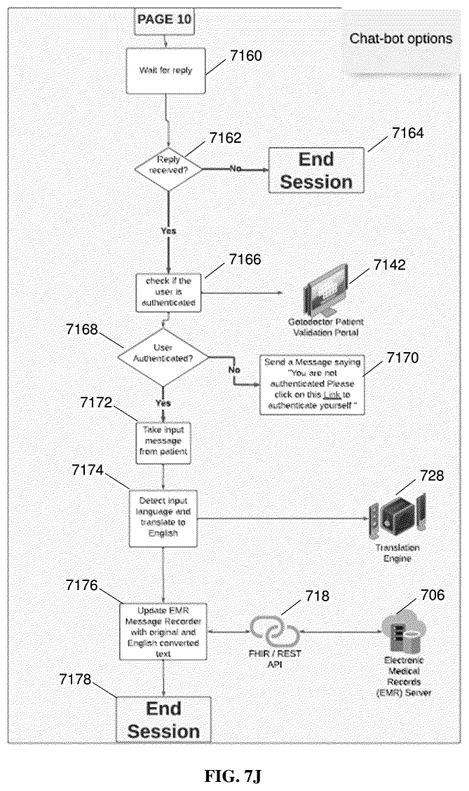

FIGS. 7A to 7J are diagrams illustrating an exemplary a mobile device translation workflow. FIG. 7A (Page 01) of the mobile device translation workflow illustrating initiation steps from the clinic. FIG. 7B (Page 02) is a workflow diagram illustrating the service engine detection and translating the message. FIG. 7C (Page 03) is a workflow diagram illustrating the service engine depicting mobile platform to initiate the message. FIG. 7D (Page 04) is a diagram illustrating the service engine workflow for SMS users. FIG. 7E (Page 05) is a diagram illustrating the service engine workflow for Android users. FIG. 7F (Page 06) is a diagram illustrating the service engine workflow for iPhone users. FIG. 7G (Page 07) is a diagram illustrating the service engine workflow for clients with no platform info in the records. FIG. 7H (Page 08) is a workflow diagram illustrating read receipt. FIG. 7I (Page 09) is a workflow diagram illustrating patient portal verification. FIG. 7J (Page 10) is a workflow diagram illustrating patient portal verification.

FIG. 7A (Page 01) illustrates the clinic user initiation a message from the EMR system. According to FIG. 7A, EMR system 700 initiates with the physician and/or clinic staff 702 login to the EMR server at step 704 and 706. Next, the physician or clinic staff 702 will initiate a mobile message at step 708. The service engine will then process the message entered at step 710. The message is then sent to a service engine domain 712 (e.g., Teledact or Gotodoctor). The workflow will then continue at FIG. 7B (Page 02).

FIG. 7B (Page 02) is a workflow diagram illustrating the service engine detection and translating the message. According to FIG. 7B (Page 02) this diagram continues with service engine processing the message entered from step 710 (from FIG. 7A, Page 01) whereby the EMR system 706 gets patient demographic and preferred language data from the EMR at step 714 using a FIHR/REST application programming interface 718 software interface. The system checks whether a preferred language is found at step 716. If no, then the system sets a default preferred language as English in the EMR, at step 720 and then sends the message to the patient in English at step 724.

According to FIG. 7B, if the preferred language is found at step 716, the system then determines whether the preferred language is English at step 722. If yes, the system sends the message to the patient in English at step 724. The workflow will then continue onto FIG. 7C (Page 03).

According to FIG. 7B, if the preferred language is not English at step 722, the service engine translates the clinical message at step 726 using translation engine 728. Thereafter, the service engine will send the message to the patient in the preferred language at step 730. The workflow will then continue onto FIG. 7C (Page 03).

FIG. 7C (Page 03) is a workflow diagram illustrating the service engine depicting mobile platform to initiate the message. According to FIG. 7C (Page 03) this diagram continues with the service engine sending the message to the patient in the preferred language or English at steps 724 or 730 (from FIG. 7B, Page 02) whereby the system checks with the records for phone support message type at step 732 by synchronizing data with the service engine 712 (e.g., Teledact or Gotodoctor) and the platform record 734. The platform record is typically stored a database. The message types may include an iMessage, Rich Content System (RCS) message and/or short message system (SMS) message.

According to FIG. 7C, the system then determines whether records are found at step 734. If no records are found, the workflow continues FIG. 7G (Page 07). If records are found at step 734, the system then determines the platform at step 738. If the platform is SMS, the workflow continues to FIG. 7D (Page 04). If the platform is Android with RCS Enabled, the workflow continues to FIG. 7E (Page 05). If the platform is iPhone (e.g., Apple), the workflow continues to FIG. 7F (Page 06).

FIG. 7D (Page 04) is a diagram illustrating the service engine workflow for SMS users. According to FIG. 7D, the system will send the message in the preferred language at step 740. A prompt message such as “Please reply ‘1” to confirm message received” may also be sent to the user. Next, the system will obtain delivery receipt at step 742. If the delivery receipt is available (or obtainable), the platform record is updated at step 744 using the FIHR/REST API interface 718 whereby the EMR record is updated at step 746. Further, the system updates the EMR's tickler/task message record to confirm that the SMS message request has been sent and received (i.e., receipt confirmation) at step 748.

According to FIG. 7D, if the delivery receipt is not obtained at step 742, the system then checks with a reply is received at step 750. If no, the system waits for the next reply for 4 hours at step 752 and the session ends at step 754. However, if a reply message is obtained at step 750, the workflow then obtains a reply at step 756. The system then checks with the reply is “1” at step 758. If the affirmative (i.e., reply is “1”), the system update the platform record at step 744. However, if the case is negative (i.e., replay is not “1”), a validation message is sent at step 760. The validation message may include “To reply, please click here to validate your phone first”. The system then determines whether patient than opens the link at step 762. If the negative (i.e., the link is not open), the workflow jumps to FIG. 7I (Page 10). If the link is opened, the system updates the EMR's tickler and task manager records, and the SMS message request is sent at step 764. Thereafter, the workflow transitions onto FIG. 7I (Page 09).

FIG. 7E (Page 05) is a diagram illustrating the service engine workflow for Android users. According to FIG. 7E, the system sends a “RCS” (Android) message in the preferred language at step 770. Next, the system checks to ensure the user is RCS enabled and reachable by the RCS Business Messaging (RBM) platform for an agent to successfully send a message. RCS Business Messaging (RBM) agents communicate with users through messages, events, and requests to achieve the user's objectives. These objectives may be simple (e.g., sending delivery notifications) or complex (e.g., booking a flight), agents use rich cards, media, and suggestions to guide users through fluid conversations that satisfy user and agent needs. According to FIG. 7E, the RBM agent who delivers the message to the destination. If the user is online, the RBM delivers the message right away, otherwise the RBM platform queues the message and delivers it when the user is next online. Furthermore, Android has an option to enable/disable the send read receipt.

According to FIG. 7E, the RCS message is then queued at step 774. The system then confirms whether the RCS message is sent at step 776. If it is not sent, then the workflow reverts to FIG. 7D (Page 04) for continuation of the process. However, if the RCS message is sent, the workflow transitions to FIG. 7H (Page 08) to confirm read receipt.

FIG. 7F (Page 06) is a diagram illustrating the service engine workflow for iPhone users. According to FIG. 7F, the system sends an “iMessage” (iPhone) message in the preferred language at step 780 using the iMessage bridge. Next, the system checks for delivery and read receipt at step 784. The system then determines whether the iMessage is sent at step 786. If it is not sent, the workflow reverts to FIG. 7D (Page 04) for continuation of the process. However, if the iMessage is sent, the workflow transitions to FIG. 7H (Page 08) to confirm read receipt.

FIG. 7G (Page 07) is a diagram illustrating the service engine workflow for clients with no platform info in records (i.e., no iOS or Android operating system). According to FIG. 7G, the RCS capability checks to see if the device is RCS-enabled (i.e., for Android) at step 790. The system then also determines whether there is RCS support at step 792. If RCS is enabled, the workflow continues to FIG. 7H (Page 08) to confirm read receipt. RCS enablement is performed by verifying API certificates 794 and Google RCS Services 796.

According to FIG. 7G, if RCS is not enabled, the system tries to send an iMessage at step 798 via an iMessage bridge 782 on an iMac computer, iPad tablet or iPhone mobile phone. Thereafter, the message is queued at step 7100 and the system then checks for delivery and read receipt at step 7102. Finally, the system then confirms whether the iMessage is sent at step 7104. If it is sent, the system continues onto FIG. 7H (Page 08) to confirm read receipt. However, if the iMessage is not sent, the system reverts to FIG. 7D (Page 04) for continuation of the process.

FIG. 7H (Page 08) is a workflow diagram illustrating read receipt. According to FIG. 7H, the workflow initiates by obtaining a delivery receipt at step 7110. The system then updates the platform record at step 7112 via the service engine 712 using the FIHR/REST API interface 718. The system also updates the EMR record at step 7114, as well as the EMR's tickler/task message recorder to indicate the message has been delivered at step 7116 via the EMR server 706.

According to FIG. 7H, after obtaining the delivery receipt at step 7110, the system will then obtain a read receipt at step 7118 where it will update the platform record 7120. Via the 7112 via the service engine 712 using the FIHR/REST API interface 718. The system also updates the EMR record at step 7122, as well as the EMR's tickler/task message recorder to indicate the message has been read at step 7124 via the EMR server 706.

According to FIG. 7H, after obtaining the read receipt at step 7118, the system will then determine whether a reply is received at step 7126. If no reply at step 7126, the session ends at step 7128. If a reply is received, a validation message is sent at step 7130. The validation message may include “To reply, please click here to validate your phone first” at step 7130. The system would then wait for next reply in the next 4 hours at step 7132 whereby the session will end at step 7128.

According to FIG. 7H, if the patient opens the link at step 7134, the workflow transitions to FIG. 7I (Page 09). If the patient does not open the link at step 7314, the workflow then transitions to FIG. 7G (Page 10).

FIG. 7I (Page 09) is a workflow diagram illustrating patient portal verification. According to FIG. 7I, the patient opens the link at step 7140 via a patient validation portal 7142. The patient fills in the health card number and date of birth (DoB) at step 7144 and the system then validates the information at step 7146 via the FIHR/REST API interface 718 with the EMR server 706. The system then checks whether the information is correct at step 7148. If no, the system loops back to step 7144 to recheck for health card number and date of birth.

According to FIG. 7I, if the information is correct at step 7148, the system authenticates the user for 24 hours to send SMS back at step 7150 via the patient validation portal 7142. The system will also show an option to change the preferred language at step 7152 and provide a list of language options 7154. An exemplary list of languages includes French, Dutch, German, Punjabi, Chinese and Spanish, however other languages may also be considered.

According to FIG. 7I, the system determines whether a language change is selected at step 7156. If a language change is selected at step 7156, the system will update the language at step 7160 via the FIHR/REST API interface 718 with the EMR server 706 whereby the system moves to step 7158. If no language change is selected at step 7156, the system sends a message to the user in the preferred language at step 7158. An exemplary message may include “The system has authenticated you. Any reply made within 24 hours to this message will be stored on the EMR for clinic to review”. Thereafter, the system continues onto FIG. 7J (Page 10) is a workflow diagram illustrating chat-bot options.

FIG. 7J (Page 10) is a workflow diagram illustrating patient chat-bot options. According to FIG. 7J, a chat-bot option initiates with the system waiting for a reply at step 7160. The system determines whether a reply is received at step 7162. If no reply, the session ends at step 7164. If a reply is received, the system checks if the user is authenticated at step 7166 via the patient validation portal.

According to FIG. 7J, if the user is not authenticated at step 7168, a confirmation message is sent at step 7170. The confirmation message may state “You are not authenticated. Please click on this link to authenticate yourself.”. However, if user authentication is successful at step 7168, the system takes the input message from the patient at step 7172.

According to FIG. 7J, the next step is to detect the input language and translate to English at step 7174 using translation engine 728. Thereafter, the system updates the EMR message recorder with the original English converted text at step 7176 via the FIHR/REST API interface 718 with the EMR server 706. Finally, the session ends at step 7178.

### Security and Authentication

In further embodiments of this disclosure, further security enhancements can be added to the communication system including features related to security and authentication. In a further embodiment, two-factor authentication may be implemented to authenticate the user to ensure that the intended user authenticates prior to receiving messages. For example, a SMS message may be sent to a phone for a user to receive a passcode (i.e., 6-digit password). The passcode will be delivered through another communication channel other than SMS (e.g., another phone number, home telephone line, email, etc.). Once the user receives the passcode and successfully enters it, the user can then download the message.

In further embodiments, a plurality of passwords may be stored in the EMR system. The patient may be requested to authenticate with one or more passwords to be able to receive SMS notification. For example, the patient may be requested to provide “their mother's maiden name”, “name of first pet”, “make of first car” or “middle name of oldest child” to be able to proceed in receiving the SMS messages.

In further embodiments, SMS notification may be classified as either administrative messages or private messages. Administrative message may include notifications to doctor's appointments (i.e., date and time) and notification to update portal info to EMR. Private messages may include actual patient results from doctor's appointments or patient medicine dosages. Administrative message may be delivered normally via SMS text messages without any security or authentication. Private messages may require 2 factor authentication (2FA) to proceed to receive and review these messages. On the EMR system portal, both administrative and private messages may be recorded, but can be classified differently and displayed in different colors or different areas of the portal dashboard.

From the portal page, messages can be categorized as administrative message or private messages (i.e., PHI messages), which in turn, is recorded and flagged in the patient's chart. This allows for easy and efficient searching of messages in the patient's chart based on the message type.

In further embodiments, a SMS message may be sent to a mobile device (i.e., mobile electronic phone or tablet). The SMS message may contain a hyperlink that when the user selects, redirects them to download a dedicated mobile application or redirects to a specific web-based portal through a web browser.

In further embodiments, a dedicated secure instant message application may be developed on the mobile device. The instant messenger application will be developed for iOS or Android. Upon receiving a SMS notification, a link will be provided to open the instant messenger application. Further authentication through the instant messenger application will ensue and upon successful authentication, the patient would be able to access the secure message.

In further embodiments, in addition to a dedicated secure instant messenger application, integration with existing Facebook Messenger, Instagram, WhatsApp. LINE, WeChat, Discord, or other popular public instant messaging applications would be considered, whereby upon successful authentication, the SMS message content may be displayed in the other instant messaging application.

In further embodiments, the EMR system may be integrated with a mobile device to enable secure exchange of messages. For example, the EMR system may identify a person via SMS by detecting the mobile operating system (e.g., iOS or Android) to send secure messages based on their phone type.

### Calendar and Scheduler

In further embodiments, the EMR system may support sending SMS or text messages at scheduled times based on clinic or patient preferences. For example, a profile preference may be set up for the patient not to receive messages outside of working hours (e.g., 9 am to 5 pm). The clinic may schedule only messages to be sent out during working hours (e.g., from 9 am to 5 pm).

In further embodiments, the EMR system may integrate SMS support with a calendar application. A calendar invite may be sent via SMS. When the user receives the SMS, they can add it to their calendar that is integrated with the EMR system.

According to further embodiments, a method of providing multi-lingual translation of EMR notification messages to the mobile device of a recipient from an EMR system is disclosed. The method comprises the steps of logging into an online account of the EMR system, creating a data message to send to a mobile platform, processing the data message using a service engine, receiving patient demographics and preferred language info from the EMR system, and determining preferred language of communication. If the preferred language is English, sending the message in English to the recipient; otherwise, if the preferred language is a second language, translating the message into the second language and sending the message in the second language to the recipient.

According to the disclosure, the method further comprises the steps of checking the system with records for mobile device support message type and if a record is not found, checking to determine whether the mobile device is an RCS-enabled Android® device. If RCS is not enabled, send iMessage®, check for delivery and read receipt. If a record is found, determine a mobile device platform and if the platform is SMS, send a SMS message, obtain delivery receipt, obtain a reply. If the platform is Android®, send an RCS message, queue the RCS message for further sending if user is offline, obtain a delivery receipt, obtain read receipt. If the platform is iOS for iPhone® devices, send iMessage®, check for delivery and read receipt, obtain delivery receipt, obtain read receipt and update EMR record.

According to the disclosure, the method further comprises patient portal for verification, a module for chat-bot support and an option to provide read receipt to the recipient. The service engine is an online portal. The option to provide read receipt to the recipient. The mobile platform is selected from a list consisting of Android®, RCS, iOS®, SMS, and email. The message type is selected from a list consisting of iMessage®, RCS, SMS, and email.

According to the disclosure, the step of checking the system with records further comprising checking the system data records. The communication with the EMR system and service engine is conducted using FIHR APIs, REST APIs and other connectivity APIs.

According to the disclosure, the method further comprises supporting two-factor authentication. The two-factor authentication further comprising receiving a SMS message passcode to the user mobile phone.

According to further embodiments, an EMR notification system for providing multi-lingual translation of EMR notification messages to the mobile device of a recipient is disclosed. The system comprises a computer to create a message, an EMR server in communication with EMR system, a pharmacy management system (PMS) in communication with EMR system, a database to store records, an application programming interface (API) connectivity module, a recipient mobile device with one or more supported platforms, a messenger module; a validation portal and a translation engine configured to translate the text messages to another language prior to delivery of the message to the recipient. The EMR notification system, in communication with the EMR server is configured to receive a message from a sender, translate the message into another language, send the message on the recipient mobile device, receive notification of read and delivery receipt of the message and update records of the EMR system with translation records and delivery status.

According to the disclosure, the recipient of the system can be patient, physician, or clinic staff. The database is configured to store demographics info and further consists of a tickler and task system module. The database selected list consisting of platform record database, pharmacy database, language preferences database, demographics database, message recorder database. The messenger module is selected form list consisting of iMessage® module, RCS Android® module and SMS module. The application programming interface (API) connectivity module further comprises FIHR API, REST APIs and other API connections. The system further comprises supporting two-factor authentication configured to receive a SMS message passcode to the user mobile phone and support for chat-bot module.

### General Considerations

Implementations disclosed herein provide systems, methods, and apparatus for generating or augmenting training data sets for machine learning training. The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code, or data that is/are executable by a computing device or processor. A “module” can be considered as a processor executing computer-readable code.

A processor as described herein can be a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, or microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. In some embodiments, a processor can be a graphics processing unit (GPU). The parallel processing capabilities of GPUs can reduce the amount of time for training and using neural networks (and other machine learning models) compared to central processing units (CPUs). In some embodiments, a processor can be an ASIC including dedicated machine learning circuitry custom-build for one or both of model training and model inference.

The disclosed or illustrated tasks can be distributed across multiple processors or computing devices of a computer system, including computing devices that are geographically distributed. The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database, or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.” While the foregoing written description of the system enables one of ordinary skill to make and use what is considered presently to be the best mode thereof, those of ordinary skill will understand and appreciate the existence of variations, combinations, and equivalents of the specific embodiment, method, and examples herein. The system should therefore not be limited by the above-described embodiment, method, and examples, but by all embodiments and methods within the scope and spirit of the system. Thus, the present disclosure is not intended to be limited to the implementations shown herein but is to be accorded the widest scope consistent with the principles and novel features disclosed herein.

What is claimed:

1. A method of providing multi-lingual translation of EMR notification messages to the mobile device of a recipient from an EMR system, the method comprising:
logging into an online account of the EMR system;
creating a data message to send to a mobile platform;
processing the data message using a service engine;
receiving patient demographics and preferred language info from the EMR system;
determining preferred language of communication; and
if the preferred language is English, sending the message in English to the recipient;
otherwise if the preferred language is a second language;
translating the message into the second language; and
sending the message in the second language to the recipient.

2. The method of claim 1 further comprising:
checking the system with records for mobile device support message type;
if a record is not found, checking to determine whether the mobile device is an RCS-enabled Android® device;
if RCS is not enabled, send using iMessage®;
check for delivery and read receipt;
if a record is found, determine a mobile device platform; and
if the platform is SMS,
send a SMS message;
obtain a delivery receipt;
obtain a reply;
if the platform is Android®,
send a RCS message;
queue the RCS message for further sending if user is offline;
obtain a delivery receipt;
obtain a read receipt;
if the platform is iOS for iPhone® devices;
send using iMessage®;
check for delivery and read receipt;
obtain a delivery receipt;
obtain a read receipt;
update the EMR record.

3. The method of claim 1 further comprising a patient portal for verification.

4. The method of claim 1 further a module for chat-bot support.

5. The method of claim 1 wherein the service engine is an online portal.

6. The method of claim 1 further comprising the option to provide read receipt to the recipient.

7. The method of claim 1 wherein the mobile platform is selected from a list consisting of Android®, RCS, iOS®, SMS, and email.

8. The method of claim 1 wherein the message type is selected from a list consisting of iMessage®, RCS, SMS and email.

9. The method of claim 1 wherein the step of checking the system with records further comprising checking the system data records.

10. The method of claim 1 wherein the communication with the EMR system and service engine is conducted using FIHR APIs, REST APIs and other connectivity APIs.

11. The method of claim 1 further comprises supporting two-factor authentication.

12. The method of claim 11 wherein the two-factor authentication further comprising receiving a SMS message passcode to the user mobile phone.

13. An EMR notification system for providing multi-lingual translation of EMR notification messages to the mobile device of a recipient, the system comprising:
a computer to create a message;
an EMR server in communication with EMR system;
a pharmacy management system (PMS) in communication with EMR system;
a database to store records;
an application programming interface (API) connectivity module;
a recipient mobile device with one or more supported platforms;
a messenger module;
a validation portal;
a translation engine configured to translate the text messages to another language prior to delivery of the message to the recipient; and
wherein the EMR notification system, in communication with the EMR server is configured to:
receive a message from a sender;
translate the message into another language;
send the message on the recipient mobile device;
receive a notification of read and delivery receipt of the message; and
update records of the EMR system with translation records and delivery status.

14. The system of claim 13 wherein the recipient can be patient, physician or clinic staff.

15. The system of claim 13 wherein the database is configured to store demographics info and further consists of a tickler and task system module.

16. The system of claim 13 wherein the database selected list consisting of platform record database, pharmacy database, language preferences database, demographics database, message recorder database.

17. The system of claim 13 wherein the messenger module is selected form list consisting of iMessage® module, RCS Android® module and SMS module.

18. The system of claim 13 wherein the application programming interface (API) connectivity module further comprises FIHR API, REST APIs and other API connections.

19. The system of claim 13 further comprising supporting two-factor authentication configured to receive a SMS message passcode to the user mobile phone.

20. The system of claim 13 further comprising a module for chat-bot support.
